(19) United States
(12) Patent Application Publication
(10) Pub. No.: US 2026/0369408 A1
(43) Pub. Date: Nov. 19, 2026

(54) DISEASE DETERMINATION DEVICE, INFORMATION PROCESSING SYSTEM, DISEASE DETERMINATION METHOD, AND PROGRAM

(71) Applicants: NEC Corporation, Tokyo (JP); TOHOKU UNIVERSITY, Sendai-shi (JP)

(72) Inventors: Ryoichi FURUKAWA, Tokyo (JP); Yusuke AKAMATSU, Tokyo (JP); Terumi UMEMATSU, Tokyo (JP); Tomohiko SHINDO, Sendai-shi (JP); Sachie KASUKABE, Sendai-shi (JP); Satoshi YASUDA, Sendai-shi (JP)

(73) Assignees: NEC Corporation, Tokyo (JP); TOHOKU UNIVERSITY, Sendai-shi (JP)

(21) Appl. No.: 19/666,551

(22) Filed: May 4, 2026

(30) Foreign Application Priority Data
May 14, 2025 (JP) 2025-081308

(51) Int. Cl. G16H 50/20; G06N 20/00; G16H 30/40; G16H 50/70

(52) U.S. Cl. CPC G16H 50/20; G06N 20/00; G16H 30/40; G16H 50/70

(57) ABSTRACT

A disease determination device acquires a plurality of site images of a living body in which an abnormality appears in a case where a predetermined disease is present. The disease determination device inputs each of the plurality of site images into a feature extraction model to extract feature information of the disease corresponding to each of the site images. The disease determination device calculates, for each of the site images, a weight value corresponding to importance of the site image for determining the disease, using the feature information corresponding to the site image. The disease determination device determines presence or absence of the disease by inputting, into a determination model for the disease, feature information obtained by multiplying the feature information corresponding to the plurality of site images by the weight value.

### CROSS-REFERENCE TO RELATED APPLICATIONS

The present application claims priority to Japanese Patent Application No. 2025-081308, filed May 14, 2025, the contents of which are incorporated herein by reference.

### BACKGROUND

### Technical Field

The present disclosure relates to a disease determination device, an information processing system, a disease determination method, and a program.

### Background Art

Research has been conducted to easily determine the presence or absence of a possibility of a predetermined disease.

In relation to the technique, for example, Japanese Unexamined Patent Application, First Publication No. 2024-51775 (hereinafter, referred to as Patent Document 1) discloses a technique for causing a computer to execute a process of inputting acquired examination information and extracted features into a learning model trained to output information related to an onset risk of ischemic heart disease, executing a calculation by the learning model, and outputting information related to the onset risk of ischemic heart disease obtained from the learning model.

### SUMMARY

More specifically, the technique of Patent Document 1 involves acquiring a tomographic image of a blood vessel and blood examination information, identifying a lesion candidate within the blood vessel, and extracting morphological features of the lesion candidate from the tomographic image. The acquired examination information and the extracted morphological features are input to a learning model, which is trained to output information regarding the onset risk of ischemic heart disease in response to input of blood examination information and the features of morphology of the lesion candidate. The model then performs calculations and outputs the resulting information related to the onset risk of ischemic heart disease.

Techniques that determine the possibility of a predetermined disease using tomographic images of blood vessels are highly invasive, as they require passing a catheter through the blood vessels. Consequently, there is a demand for an ischemic heart disease determination device that can perform determinations simply and non-invasively for users. Such a device would acquire images of parts of the living body surface that are presumed to have a strong correlation with ischemic heart disease, which can be captured using a camera, thereby providing a more convenient and non-invasive alternative to related techniques.

An example object of the present disclosure is to provide a disease determination device, an information processing system, a disease determination method, and a program that solve the problems mentioned above.

A disease determination device according to one example aspect of the present disclosure includes: a memory configured to store instructions; and a processor configured to execute the instructions to: acquiring a plurality of site images of a living body in which an abnormality appears in a case where a predetermined disease is present; inputting each of the plurality of site images into a feature extraction model to extract feature information of the disease corresponding to each of the site images; calculating, for each of the site images, a weight value corresponding to importance of the site image for determining the disease, using the feature information corresponding to the site image; and determining presence or absence of the disease by inputting, into a determination model for the disease, feature information obtained by multiplying the feature information corresponding to the plurality of site images by the weight value.

An information processing system according to one example aspect of the present disclosure includes: a memory configured to store instructions; and a processor configured to execute the instructions to: acquiring a plurality of site images of a living body in which an abnormality appears in a case where a predetermined disease is present; inputting each of the plurality of site images into a feature extraction model to extract feature information of the disease corresponding to each of the site images; calculating, for each of the site images, a weight value corresponding to importance of the site image for determining the disease, using the feature information corresponding to the site image; and determining presence or absence of the disease by inputting, into a determination model for the disease, feature information obtained by multiplying the feature information corresponding to the plurality of site images by the weight value.

A disease determination method according to one example aspect of the present disclosure includes: acquiring a plurality of site images of a living body in which an abnormality appears in a case where a predetermined disease is present; inputting each of the plurality of site images into a feature extraction model to extract feature information of the disease corresponding to each of the site images; calculating, for each of the site images, a weight value corresponding to importance of the site image for determining the disease, using the feature information corresponding to the site image; and determining presence or absence of the disease by inputting, into a determination model for the disease, feature information obtained by multiplying the feature information corresponding to the plurality of site images by the weight value.

A program according to one example aspect of the present disclosure causes a computer to execute: acquiring a plurality of site images of a living body in which an abnormality appears in a case where a predetermined disease is present; inputting each of the plurality of site images into a feature extraction model to extract feature information of the disease corresponding to each of the site images; calculating, for each of the site images, a weight value corresponding to importance of the site image for determining the disease, using the feature information corresponding to the site image; and determining presence or absence of the disease by inputting, into a determination model for the disease, feature information obtained by multiplying the feature information corresponding to the plurality of site images by the weight value.

### BRIEF DESCRIPTION OF THE DRAWINGS

FIG. 1 is a schematic configuration diagram of an information processing system according to one example aspect of the present disclosure.

FIG. 2 is a diagram showing an example of a hardware configuration of a disease determination device according to one example aspect of the present disclosure.

FIG. 3 is a functional block diagram of the disease determination device according to one example aspect of the present disclosure.

FIG. 4 is a first diagram showing a processing overview of the disease determination device according to one example aspect of the present disclosure.

FIG. 5 is a diagram showing a processing flow of the disease determination device according to one example aspect of the present disclosure.

FIG. 6 is a second diagram showing a processing overview of the disease determination device according to one example aspect of the present disclosure.

FIG. 7 is a functional block diagram of the disease determination device according to another example aspect of the present disclosure.

FIG. 8 is a diagram showing a processing flow of the disease determination device according to another example aspect of the present disclosure.

### EMBODIMENTS

Hereinafter, a disease determination device, an information processing system, a disease determination method, and a program according to the present disclosure will be described, with reference to the drawings.

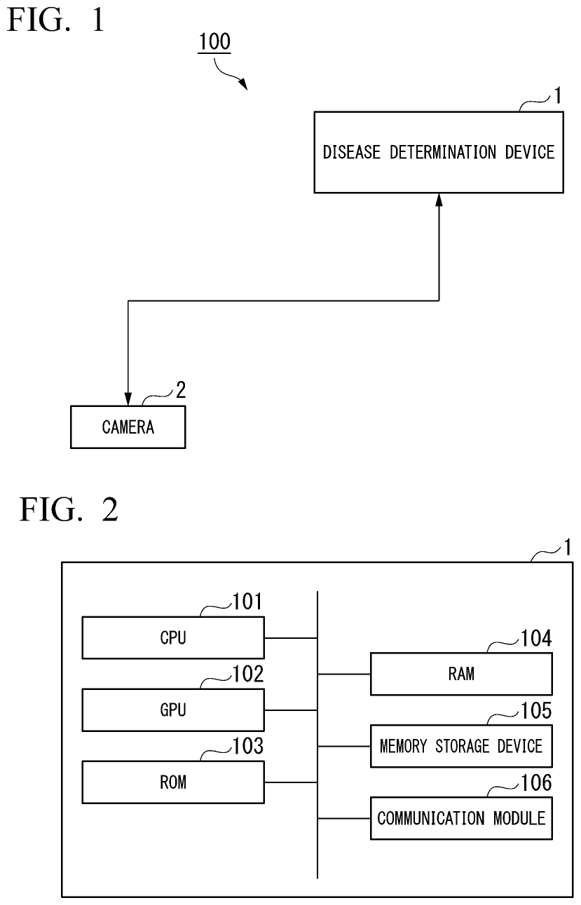

FIG. 1 is a schematic configuration diagram of an information processing system according to one example aspect of the present disclosure.

An information processing system 100 includes at least a disease determination device 1. The information processing system 100 may include a camera 2. The camera 2 captures living body images of a subject (for example, a subject such as a patient) and transmits the living body images to the disease determination device 1. In the present disclosure, living body images will be described centering on site images capturing a head (corresponding to a predetermined part) including a face, but may be site images capturing a part of a living body surface (corresponding to a predetermined site) other than the head. In other example aspects, X-ray images, CT (Computed Tomography) images, MRI (Magnetic Resonance Imaging) images, or the like may be used as the site images.

FIG. 2 is a diagram showing an example of a hardware configuration of the disease determination device according to one example aspect of the present disclosure. The disease determination device 1 is, as an example, as shown in FIG. 2, a computer including hardware such as a CPU (Central Processing Unit) 101, a GPU (Graphics Processing Unit) 102, a ROM (Read Only Memory) 103, a RAM (Random Access Memory) 104, a storage device 105 such as another HDD (Hard Disk Drive), a communication module 106, and so forth. The CPU 101 of the disease determination device 1 executes a disease determination program stored in the storage device 105, thereby performing the functions of each processing unit described below. The disease determination device 1 may exhibit the functions of each processing unit described below by one computer. Alternatively, multiple computers may execute programs corresponding to functions of one or multiple processing units among the processing units, and the computers may cooperate to operate as an information processing system that is a computer system corresponding to the disease determination device 1.

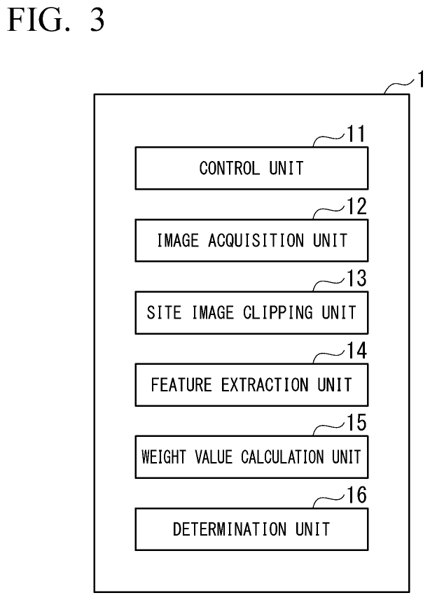

FIG. 3 is a functional block diagram of the disease determination device according to one example aspect of the present disclosure.

The disease determination device 1 exhibits the functions of a control unit 11, an image acquisition unit 12, a site image clipping unit 13, a feature extraction unit 14, a weight value calculation unit 15, and a determination unit 16 by executing the disease determination program on the computer.

The control unit 11 controls each processing unit.

The image acquisition unit 12 acquires images for use in disease determination.

The site image clipping unit 13 clips multiple site images, used for disease determination, from the images acquired by the image acquisition unit 12.

The feature extraction unit 14 inputs each of the multiple site images into a feature extraction model and extracts feature information corresponding to each of the site images.

The weight value calculation unit 15 calculates weight values for the respective site images based on their importance in disease determination, using the feature information corresponding to each site image.

The determination unit 16 determines the presence or absence of the disease by inputting feature information obtained by multiplying the feature information of the site images by the calculated weight values into a disease determination model.

In the present disclosure, the predetermined disease determined by the disease determination device 1 is ischemic heart disease. The captured images obtained by the image acquisition unit 12 include a front face captured image capturing a face included in a head from a front, a left-side face captured image capturing a left side of the face, a right-side face captured image capturing a right side of the face, and a top-of-head captured image capturing a top of the head. Moreover, the site images obtained from the site image clipping unit 13 include a left ear captured image obtained by clipping out around a left earlobe from the left-side face captured image, a right ear captured image clipped out from the right-side face captured image, and a left eye captured image and a right eye captured image obtained by clipping out eyes and skin around the eyes clipped out from the front face captured image. In other words, the disease determination device 1 of the present disclosure determines the disease using eight site images: the left ear captured image, the right ear captured image, the left eye captured image, the right eye captured image, the front face captured image, the left-side face captured image, the right-side face captured image, and the top-of-head captured image.

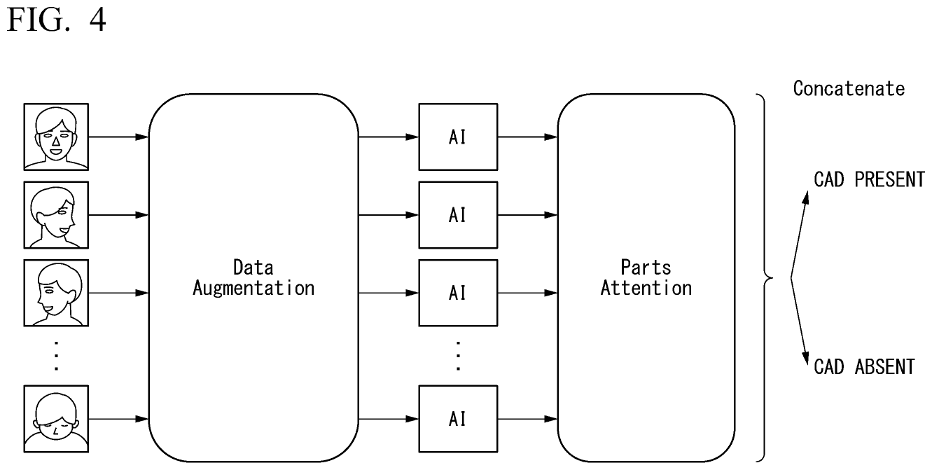

FIG. 4 is a first diagram showing a processing overview of the disease determination device according to one embodiment of the present disclosure.

The disease determination device 1 according to the present disclosure uses, as the multiple site images of the living body, as described above, a left ear captured image, a right ear captured image, a left eye captured image, a right eye captured image, a front face captured image, a left-side face captured image, a right-side face captured image, and a top-of-head captured image. More specifically, using the left-side face captured image, the right-side face captured image, and the front face captured image among the four head images, namely, the front face captured image, the left-side face captured image, the right-side face captured image, and the top-of-head captured image which are acquired by the image acquisition unit 12 of the disease determination device 1, the site image clipping unit 13 clips out the left ear captured image, the right ear captured image, the left eye captured image, and the right eye captured image.

Then, the feature extraction unit 14 of the disease determination device 1 performs data augmentation processing on each of the left ear captured image, the right ear captured image, the left eye captured image, the right eye captured image, the front face captured image, the left-side face captured image, the right-side face captured image, and the top-of-head captured image. In the data augmentation processing, image processing such as horizontal inversion, color transformation, and brightness transformation is similarly performed on each of the site images of the left ear captured image, the right ear captured image, the left eye captured image, the right eye captured image, the front face captured image, the left-side face captured image, the right-side face captured image, and the top-of-head captured image to generate transformed images subjected to each transformation.

The feature extraction unit 14 of the disease determination device 1 inputs the left ear captured image, the right ear captured image, the left eye captured image, the right eye captured image, the front face captured image, the left-side face captured image, the right-side face captured image, and the top-of-head captured image, as well as transformed images after multiple different data augmentation transformations, into a feature extraction model to calculate feature information indicating a disease corresponding to each image. The feature extraction model is, as an example, a machine learning model constructed using ResNet18 (a convolutional neural network having a depth of 18 layers, which is a part of the Residual Network (ResNet) family). ResNet18 may use weights pre-trained by public data called ImageNet 1K as initial values, or may be trained from initial weights without using them. Moreover, a CNN (Convolutional Neural Network) other than ResNet18 may be used for the feature extraction model, or a network based on ViT (Vision Transformer) may be used. The feature extraction unit 14 outputs the feature information using up to ResBlock4 (indicating a fourth residual block (Residual Block) of the ResNet18 architecture) in the ResNet18.

As parts attention processing (Parts Attention), the weight value calculation unit 15 calculates a weight value corresponding to each site image using each feature information of the transformed image corresponding to each site image acquired from the feature extraction unit 14. The weight value corresponding to each site image indicates a weight value of each of the captured site images of a subject such as a patient, and the weight value calculation unit 15 calculates each weight value such that a larger weight value is assigned to a site image considered important for disease determination. Thereafter, the determination unit 16 of the disease determination device 1 inputs values obtained by multiplying features extracted by the feature extraction unit 14 from each site image by the weight values into the determination model to output a determination result of the presence or absence of the disease.

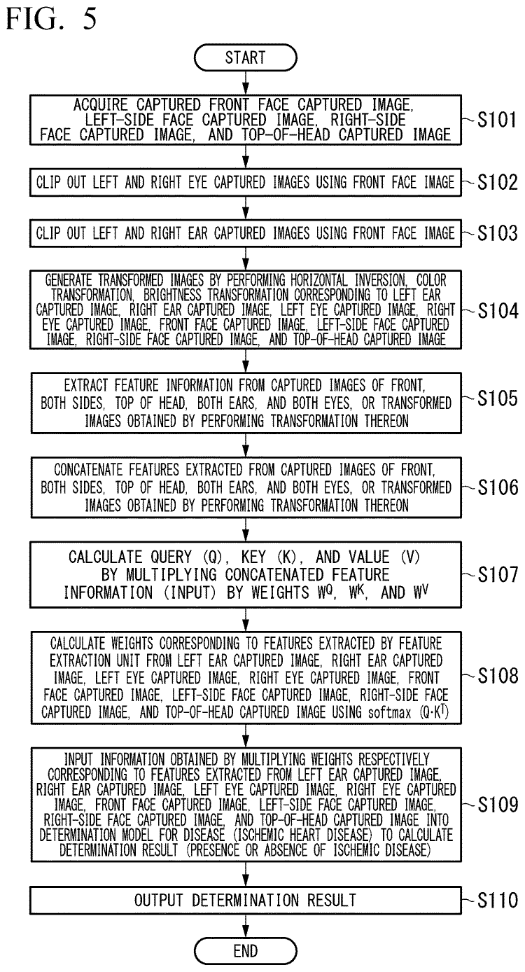

FIG. 5 is a diagram showing a processing flow of the disease determination device according to one example aspect of the present disclosure.

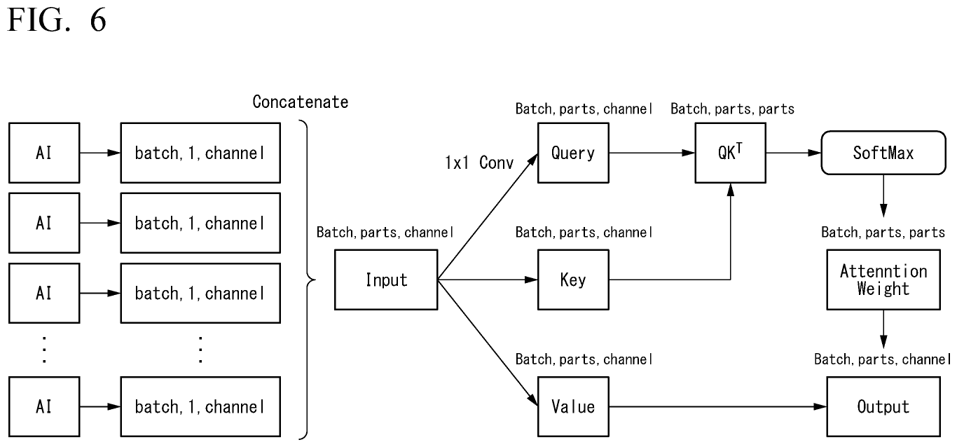

FIG. 6 is a second diagram showing a processing overview of the disease determination device according to one example aspect of the present disclosure.

Next, details of processing of the disease determination device will be described.

The image acquisition unit 12 of the disease determination device 1 acquires a front face captured image, a left-side face captured image, a right-side face captured image, and a top-of-head captured image from the camera 2 (Step S101). A site image clipping unit 13 clips out a left eye captured image and a right eye captured image capturing eyes and skin around the eyes using the front face image (Step S102). Clipping out of the eye captured images by the site image clipping unit 13 may be performed, for example, by recognizing eyeballs by pattern recognition or the like and then clipping out a range such as a rectangle of a predetermined size centered on the eyeballs. The site image clipping unit 13 clips out left and right ear captured images capturing around earlobes using the left-side face captured image and the right-side face captured image (Step S103). Clipping out of the left and right ear captured images by the site image clipping unit 13 may be performed, for example, by recognizing ears by pattern recognition or the like and then clipping out a range such as a rectangle of a predetermined size centered on the earlobes of the ears. The site image clipping unit 13 outputs the left ear captured image, the right ear captured image, the left eye captured image, the right eye captured image, the front face captured image, the left-side face captured image, the right-side face captured image, and the top-of-head captured image to the feature extraction unit 14.

The feature extraction unit 14 performs data augmentation by applying multiple distinct transformations, such as horizontal inversion, color transformation, and brightness transformation, to the left ear captured image, the right ear captured image, the left eye captured image, the right eye captured image, the front face captured image, the left-side face captured image, the right-side face captured image, and the top-of-head captured image, and generates images (transformed images) that correspond to the left ear captured image, the right ear captured image, the left eye captured image, the right eye captured image, the front face captured image, the left-side face captured image, the right-side face captured image, and the top-of-head captured image and to which the horizontal inversion, the color transformation, the brightness transformation, and the like have been performed (Step S104).

The feature extraction unit 14 inputs each of the left ear captured image, the right ear captured image, the left eye captured image, the right eye captured image, the front face captured image, the left-side face captured image, the right-side face captured image, the top-of-head captured image, or transformed images obtained by performing multiple transformations (horizontal inversion, color transformation, brightness transformation, and so forth) thereon into the feature extraction model to extract respective feature information (Step S105). Examples of targets of the feature information include abnormalities (dermadromes) such as wrinkles, spots, skin color (yellowing, acanthosis nigricans, and so forth), macules, erythema, warts, eruptions, boils, lumps, and capillaries. The feature extraction model may be a learning model generated for each image. More specifically, the feature extraction model is a machine learning model trained to output feature information specific to the onset of a disease (ischemic heart disease) based on an input image, such as an image of a subject such as a patient or a transformed image thereof, when the disease (ischemic heart disease) is present. The feature extraction unit 14 outputs the feature information of the eight site images or the transformed images subjected to the multiple transformations to the weight value calculation unit 15.

The weight value calculation unit 15 concatenates the respective feature information extracted by the feature extraction unit 14 from the left ear captured image, the right ear captured image, the left eye captured image, the right eye captured image, the front face captured image, the left-side face captured image, the right-side face captured image, the top-of-head captured image, or images after transformation obtained by performing multiple transformations on these images (Step S106). It should be noted that the respective feature information extracted by the feature extraction unit 14 from any multiple images among the left ear captured image, the right ear captured image, the left eye captured image, the right eye captured image, the front face captured image, the left-side face captured image, the right-side face captured image, the top-of-head captured image, and the images after transformation obtained by performing multiple transformations on these images may be concatenated. The weight value calculation unit 15 multiplies the concatenated feature information (Input) by weights W^{Q}, W^{K}, and W^{V} to calculate Query (Q), Key (K), and Value (V) (Step S107). Query, Key, and Value can be calculated by the following Equation (1), Equation (2), and Equation (3), respectively. It should be noted that 1×1Conv indicates 1×1 Convolution (1×1 convolution processing).

Q=Input·W^{Q}, (W^{Q}: Weights of 1×1 Conv for transforming into Q (Equation (1))

K=Input·W^{K}, (W^{K}: Weights of 1×1 Conv for transforming into K (Equation (2))

V=Input·W^{V}, (W^{V}: Weights of 1×1 Conv for transforming into V (Equation (3))

Moreover, the weight value calculation unit 15 calculates weights corresponding to features extracted by the feature extraction unit 14 from the left ear captured image, the right ear captured image, the left eye captured image, the right eye captured image, the front face captured image, the left-side face captured image, the right-side face captured image, and the top-of-head captured image using softmax(Q·K^{T}) of the following Equation (4) (Step S108).

Output=softmax(Q·K^{T})·V (Equation (4))

softmax denotes a known softmax function. Furthermore, in Equation (4), T denotes a transpose.

According to the processing of the weight value calculation unit 15 as described above, weight values of respective site images that are important for determination of the disease can be calculated using feature information corresponding to each of the site images. Moreover, although the weight calculation method used in the weight value calculation unit 15 in this case is a method based on Self Attention, it may be obtained by a method based on SENet (Squeeze and Excitation Network).

The weight value calculation unit 15 outputs, to the determination unit 16, an Output of features (Equation (4)) obtained by multiplying the calculated weights with features extracted by the feature extraction unit 14 from the left ear captured image, the right ear captured image, the left eye captured image, the right eye captured image, the front face captured image, the left-side face captured image, the right-side face captured image, and the top-of-head captured image.

The determination unit 16 inputs input information, obtained by multiplying weight values corresponding to the respective features extracted by the feature extraction unit 14 from the left ear captured image, the right ear captured image, the left eye captured image, the right eye captured image, the front face captured image, the left-side face captured image, the right-side face captured image, and the top-of-head captured image, into the determination model for the disease (ischemic heart disease). The determination model for the disease (ischemic heart disease) is a machine learning model that has been trained to learn a relationship between input information and the presence or absence of the disease (ischemic heart disease), and outputs the presence or absence of the disease based on input of the input information. The determination unit 16 inputs the input information, obtained by multiplying weight values corresponding to the respective features extracted by the feature extraction unit 14 from the left ear captured image, the right ear captured image, the left eye captured image, the right eye captured image, the front face captured image, the left-side face captured image, the right-side face captured image, and the top-of-head captured image, into the determination model for the disease (ischemic heart disease) to calculate a determination result (presence or absence of ischemic disease) (Step S109), and outputs the determination result to a predetermined output destination (Step S110). Moreover, clinical record information used when a physician diagnoses ischemic heart disease, such as gender, age, chest pain, medical history, smoking history, diabetes, hypertension, history of dyslipidemia, and family history, may be input to the determination model.

As described above, one example aspect of the present disclosure has been described. According to the processing described above, not only a front face captured image, a left-side face captured image, a right-side face captured image, and a top-of-head captured image are used, but also ear captured images and eye captured images, which are site images in which features strongly correlated to the disease appear and from which age and gender are difficult to estimate are used. There is a correlation between a risk of onset of ischemic heart disease and indicators such as age and gender (the likelihood of the disease is higher in males than in females), and when an image including an entire face is input, there is a high likelihood that information such as age and gender is extracted. Therefore, in the case where only images including an entire face, such as a front face captured image, a left-side face captured image, a right-side face captured image, and a top-of-head captured image, are used, there is a high likelihood that determination of the presence or absence of ischemic heart disease is performed based only on age and gender rather than on features specific to the disease. For this reason, in addition to images from which age and gender can be estimated, such as a front face captured image, a left-side face captured image, a right-side face captured image, and a top-of-head captured image, ear captured images and eye captured images from which age and gender are difficult to estimate are also used, whereby a determination result indicating the presence or absence of ischemic heart disease that does not depend solely on indicators such as age and gender can be calculated.

According to the processing described above, weight values for respective site images that are important for determining the disease are calculated using features corresponding to each site image. According to this weight calculation, larger weight values are calculated for site images in which the influence of the disease appears more strongly. Therefore, it is possible to determine the presence or absence of the predetermined disease while strongly reflecting the influence of features that are considered to have a strong correlation with the disease for a subject person such as a patient. Furthermore, since the site images are obtained from images captured by a general-purpose camera 2, there is no burden on the subject, and non-invasiveness can be ensured.

In the processing described above, an example in which the disease is an ischemic disease is described. However, the disease may be another disease, for example, an arteriosclerotic disease other than ischemic heart disease, such as an aortic aneurysm, cerebral infarction, or arteriosclerosis obliterans of a lower limb, and the disease determination device 1 may determine the presence or absence of a possibility of such other arteriosclerotic diseases. In such a case, the site images may include images other than a left ear captured image, a right ear captured image, a left eye captured image, a right eye captured image, a front face captured image, a left-side face captured image, a right-side face captured image, and a top-of-head captured image. For example, instead of face images, site images of predetermined sites of a living body surface other than the face (for example, a neck, a chest, an abdomen, a lower abdomen, or a leg) may be used, and combinations with face images or combinations of multiple sites may also be used. Furthermore, the disease may be a disease other than an arteriosclerotic disease (for example, organ failure such as heart failure or renal failure). In a case of organ failure, site images such as an eye captured image, a front face captured image, a left-side face captured image, and a right-side face captured image may be used, and swelling, pigmentation, spots, and the like may be targets of feature information. It should be noted that, from a viewpoint of low invasiveness, site images of predetermined sites extracted from X-ray images, CT images, or MRI images may also be used or used in combination.

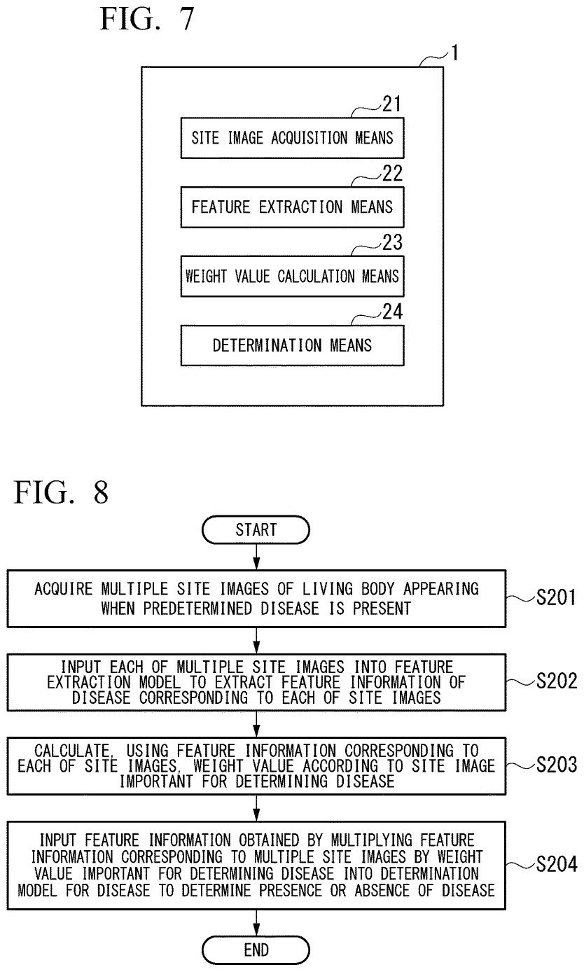

FIG. 7 is a functional block diagram of the disease determination device according to another example aspect of the present disclosure.

FIG. 8 is a diagram showing a processing flow of the disease determination device according to another example aspect of the present disclosure.

The disease determination device 1 may include, at least, a site image acquisition means 21, a feature information calculation means 22, a weight value calculation means 23, and a determination means 24.

The site image acquisition means 21 acquires multiple site images of a living body that appear when a predetermined disease is present (Step S201).

The feature information extraction means 22 inputs each of the multiple site images into a feature extraction model and extracts disease-related feature information corresponding to each site image (Step S202).

The weight value calculation means 23 calculates, for each site image, a weight value that is important for determining the disease by using the feature information corresponding to the site image (Step S203).

The determination means 24 determines the presence or absence of the disease by inputting, into a determination model for the disease, feature information obtained by multiplying the feature information corresponding to the multiple site images by the weight value (Step S204).

According to the above one example aspect, the user of the device can easily and non-invasively determine whether or not there is a possibility of disease.

While the present disclosure has been described above with reference to the example embodiments, the present disclosure is not limited to the example embodiments described above.

The whole or part of the example embodiments disclosed above can be described as, but not limited to, the following supplementary notes.

### Supplementary Note 1

A disease determination device comprising:
a site image acquisition means that acquires a plurality of site images of a living body that appear in a case where a predetermined disease is present;
a feature information extraction means that inputs each of the plurality of site images into a feature extraction model and extracts feature information of the disease corresponding to each of the site images;
a weight value calculation means that calculates, using the feature information corresponding to each of the site images, a weight value corresponding to the site image that is important for determining the disease; and
a determination means that determines the presence or absence of the disease by inputting, into a determination model for the disease, feature information obtained by multiplying the feature information corresponding to the plurality of site images by the weight value.

### Supplementary Note 2

The disease determination device according to supplementary note 1, wherein
the predetermined disease is ischemic heart disease, and
the site images include a head captured image obtained by capturing a head, an ear captured image obtained by capturing an area around an earlobe, and an eye captured image obtained by capturing an eye and skin around the eye.

### Supplementary Note 3

The disease determination device according to supplementary note 2, wherein
the head captured image includes an image obtained by capturing a face included in the head from the front, images obtained by capturing the face from left and right sides, respectively, and an image obtained by capturing a top of the head.

### Supplementary Note 4

The disease determination device according to any one of supplementary notes 1 to 3, wherein
the feature extraction model is a machine learning model that is trained to learn a relationship between the site images and feature information in the site images in a case where the disease is present, and outputs the feature information specific to onset of the disease based on input of the site images.

### Supplementary Note 5

The disease determination device according to any one of supplementary notes 1 to 4, wherein
the determination model is a machine learning model that is trained to learn a relationship between the input information and the presence or absence of the disease, and outputs the presence or absence of the disease based on input of the input information.

### Supplementary Note 6

The disease determination device according to any one of supplementary notes 1 to 5, comprising
a data augmentation means that generates a plurality of data augmented images by performing a plurality of different data augmentations on the site images, wherein
the feature information extraction means calculates feature information corresponding to each of the data augmented images by inputting each of the data augmented images into the corresponding feature extraction model, and
the weight value calculation means calculates a weight value corresponding to the site image that is important for determining the disease by combining the feature information corresponding to each of the data augmented images.

### Supplementary Note 7

An information processing system comprising:
a site image acquisition means that acquires a plurality of site images of a living body that appear in a case where a predetermined disease is present;
a feature information extraction means that inputs each of the plurality of site images into a feature extraction model and extracts feature information of the disease corresponding to each of the site images;
a weight value calculation means that calculates, using the feature information corresponding to each of the site images, a weight value corresponding to the site image that is important for determining the disease; and
a determination means that determines the presence or absence of the disease by inputting, into a determination model for the disease, feature information obtained by multiplying the feature information corresponding to the plurality of site images by the weight value.

### Supplementary Note 8

The information processing system according to supplementary note 7, wherein
the predetermined disease is ischemic heart disease, and
the site images include a head captured image obtained by capturing a head, an ear captured image obtained by capturing an area around an earlobe, and an eye captured image obtained by capturing an eye and skin around the eye.

### Supplementary Note 9

The information processing system according to supplementary note 8, wherein
the head captured image includes an image obtained by capturing a face included in the head from the front, images obtained by capturing the face from left and right sides, respectively, and an image obtained by capturing a top of the head.

### Supplementary Note 10

The information processing system according to any one of supplementary notes 7 to 9, wherein
the feature extraction model is a machine learning model that is trained to learn a relationship between the site images and feature information in the site images in a case where the disease is present, and outputs the feature information specific to onset of the disease based on input of the site images.

### Supplementary Note 11

The information processing system according to any one of supplementary notes 7 to 10, wherein
the determination model is a machine learning model that is trained to learn a relationship between the input information and the presence or absence of the disease, and outputs the presence or absence of the disease based on input of the input information.

### Supplementary Note 12

The information processing system according to any one of supplementary notes 7 to 11, comprising
a data augmentation means that generates a plurality of data augmented images by performing a plurality of different data augmentations on the site images, wherein
the feature information extraction means calculates feature information corresponding to each of the data augmented images by inputting each of the data augmented images into the corresponding feature extraction model, and
the weight value calculation means calculates a weight value corresponding to the site image that is important for determining the disease by combining the feature information corresponding to each of the data augmented images.

### Supplementary Note 13

A disease determination method comprising:
acquiring a plurality of site images of a living body that appear in a case where a predetermined disease is present;
inputting each of the plurality of site images into a feature extraction model and extracting feature information of the disease corresponding to each of the site images;
calculating, using the feature information corresponding to each of the site images, a weight value corresponding to the site image that is important for determining the disease; and
determining the presence or absence of the disease by inputting, into a determination model for the disease, feature information obtained by multiplying the feature information corresponding to the plurality of site images by the weight value.

### Supplementary Note 14

The disease determination method according to supplementary note 13, wherein
the predetermined disease is ischemic heart disease, and
the site images include a head captured image obtained by capturing a head, an ear captured image obtained by capturing an area around an earlobe, and an eye captured image obtained by capturing an eye and skin around the eye.

### Supplementary Note 15

The disease determination method according to supplementary note 14, wherein
the head captured image includes an image obtained by capturing a face included in the head from the front, images obtained by capturing the face from left and right sides, respectively, and an image obtained by capturing a top of the head.

### Supplementary Note 16

The disease determination method according to any one of supplementary notes 13 to 15, wherein
the feature extraction model is a machine learning model that is trained to learn a relationship between the site images and feature information in the site images in a case where the disease is present, and outputs the feature information specific to onset of the disease based on input of the site images.

### Supplementary Note 17

The disease determination method according to any one of supplementary notes 13 to 16, wherein
the determination model is a machine learning model that is trained to learn a relationship between the input information and the presence or absence of the disease, and outputs the presence or absence of the disease based on input of the input information.

### Supplementary Note 18

The disease determination method according to any one of supplementary notes 13 to 17, comprising
generating a plurality of data augmented images by performing a plurality of different data augmentations on the site images;
calculating feature information corresponding to each of the data augmented images by inputting each of the data augmented images into the corresponding feature extraction model; and
calculating a weight value corresponding to the site image that is important for determining the disease by combining the feature information corresponding to each of the data augmented images.

### Supplementary Note 19

A program that causes
a computer to function as:
a site image acquisition means that acquires a plurality of site images of a living body that appear in a case where a predetermined disease is present;
a feature information extraction means that inputs each of the plurality of site images into a feature extraction model and extracts feature information of the disease corresponding to each of the site images;
a weight value calculation means that calculates, using the feature information corresponding to each of the site images, a weight value corresponding to the site image that is important for determining the disease; and
a determination means that determines the presence or absence of the disease by inputting, into a determination model for the disease, feature information obtained by multiplying the feature information corresponding to the plurality of site images by the weight value.

### Supplementary Note 20

The program according to supplementary note 19, wherein
the predetermined disease is ischemic heart disease, and
the site images include a head captured image obtained by capturing a head, an ear captured image obtained by capturing an area around an earlobe, and an eye captured image obtained by capturing an eye and skin around the eye.

### Supplementary Note 21

The program according to supplementary note 20, wherein
the head captured image includes an image obtained by capturing a face included in the head from the front, images obtained by capturing the face from left and right sides, respectively, and an image obtained by capturing a top of the head.

### Supplementary Note 22

The program according to any one of supplementary notes 19 to 21, wherein
the feature extraction model is a machine learning model that is trained to learn a relationship between the site images and feature information in the site images in a case where the disease is present, and outputs the feature information specific to onset of the disease based on input of the site images.

### Supplementary Note 23

The program according to any one of supplementary notes 19 to 22, wherein
the determination model is a machine learning model that is trained to learn a relationship between the input information and the presence or absence of the disease, and outputs the presence or absence of the disease based on input of the input information.

### Supplementary Note 24

The program according to any one of supplementary notes 19 to 23, that
functions as a data augmentation means that generates a plurality of data augmented images by performing a plurality of different data augmentations on the site images, and
the feature information extraction means calculates feature information corresponding to each of the data augmented images by inputting each of the data augmented images into the corresponding feature extraction model, and
the weight value calculation means calculates a weight value corresponding to the site image that is important for determining the disease by combining the feature information corresponding to each of the data augmented images.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A disease determination device comprising:
a memory configured to store instructions; and
a processor configured to execute the instructions to:
acquiring a plurality of site images of a living body in which an abnormality appears in a case where a predetermined disease is present;
inputting each of the plurality of site images into a feature extraction model to extract feature information of the disease corresponding to each of the site images;
calculating, for each of the site images, a weight value corresponding to importance of the site image for determining the disease, using the feature information corresponding to the site image; and
determining presence or absence of the disease by inputting, into a determination model for the disease, feature information obtained by multiplying the feature information corresponding to the plurality of site images by the weight value.

2. The disease determination device according to claim 1,
wherein the predetermined disease is ischemic heart disease, and
wherein the site images include a head captured image obtained by capturing a head, an ear captured image obtained by capturing an area around an earlobe, and an eye captured image obtained by capturing an eye and skin around the eye.

3. The disease determination device according to claim 2, wherein the head captured image includes an image obtained by capturing a face included in the head from the front, images obtained by capturing the face from left and right sides, respectively, and an image obtained by capturing a top of the head.

4. The disease determination device according to claim 2, wherein the feature extraction model is a machine learning model that is trained to learn a relationship between the site images and feature information in the site images in a case where the disease is present, and that outputs the feature information specific to onset of the disease based on input of the site images.

5. The disease determination device according to claim 3, wherein the determination model is a machine learning model that is trained to learn a relationship between input information and presence or absence of the disease, and that outputs presence or absence of the disease based on input of the input information.

6. The disease determination device according to claim 5,
wherein the processor is configured to execute the instructions to generate a plurality of data augmented images by performing a plurality of different data augmentations on the site images,
wherein the processor is configured to execute the instructions to calculate feature information corresponding to each of the data augmented images by inputting each of the data augmented images into the corresponding feature extraction model, and
wherein the processor is configured to execute the instructions to concatenate the feature information corresponding to each of the data augmented images and calculate a weight value corresponding to importance of the concatenated feature information for determining the disease.

7. An information processing system comprising:
a memory configured to store instructions; and
a processor configured to execute the instructions to:
acquiring a plurality of site images of a living body in which an abnormality appears in a case where a predetermined disease is present;
inputting each of the plurality of site images into a feature extraction model to extract feature information of the disease corresponding to each of the site images;
calculating, for each of the site images, a weight value corresponding to importance of the site image for determining the disease, using the feature information corresponding to the site image; and
determining presence or absence of the disease by inputting, into a determination model for the disease, feature information obtained by multiplying the feature information corresponding to the plurality of site images by the weight value.

8. The information processing system according to claim 7,
wherein the predetermined disease is ischemic heart disease, and
wherein the site images include a head captured image obtained by capturing a head, an ear captured image obtained by capturing an area around an earlobe, and an eye captured image obtained by capturing an eye and skin around the eye.

9. The information processing system according to claim 7, wherein the head captured image includes an image obtained by capturing a face included in the head from the front, images obtained by capturing the face from left and right sides, respectively, and an image obtained by capturing a top of the head.

10. The information processing system according to claim 8, wherein the feature extraction model is a machine learning model that is trained to learn a relationship between the site images and feature information in the site images in a case where the disease is present, and that outputs the feature information specific to onset of the disease based on input of the site images.

11. The information processing system according to claim 9, wherein the determination model is a machine learning model that is trained to learn a relationship between input information and presence or absence of the disease, and that outputs presence or absence of the disease based on input of the input information.

12. The information processing system according to claim 11,
wherein the processor is configured to execute the instructions to generate a plurality of data augmented images by performing a plurality of different data augmentations on the site images,
wherein the processor is configured to execute the instructions to calculate feature information corresponding to each of the data augmented images by inputting each of the data augmented images into the corresponding feature extraction model, and
wherein the processor is configured to execute the instructions to concatenate the feature information corresponding to each of the data augmented images and calculate a weight value corresponding to importance of the concatenated feature information for determining the disease.

13. A disease determination method comprising:
acquiring a plurality of site images of a living body in which an abnormality appears in a case where a predetermined disease is present;
inputting each of the plurality of site images into a feature extraction model to extract feature information of the disease corresponding to each of the site images;
calculating, for each of the site images, a weight value corresponding to importance of the site image for determining the disease, using the feature information corresponding to the site image; and
determining presence or absence of the disease by inputting, into a determination model for the disease, feature information obtained by multiplying the feature information corresponding to the plurality of site images by the weight value.

14. The disease determination method according to claim 13,
wherein the predetermined disease is ischemic heart disease, and
wherein the site images include a head captured image obtained by capturing a head, an ear captured image obtained by capturing an area around an earlobe, and an eye captured image obtained by capturing an eye and skin around the eye.

15. The disease determination method according to claim 13, wherein the head captured image includes an image obtained by capturing a face included in the head from the front, images obtained by capturing the face from left and right sides, respectively, and an image obtained by capturing a top of the head.

16. The disease determination method according to claim 14, wherein the feature extraction model is a machine learning model that is trained to learn a relationship between the site images and feature information in the site images in a case where the disease is present, and that outputs the feature information specific to onset of the disease based on input of the site images.

17. The disease determination method according to claim 15, wherein the determination model is a machine learning model that is trained to learn a relationship between input information and presence or absence of the disease, and that outputs presence or absence of the disease based on input of the input information.

18. The disease determination method according to claim 17, further comprising:
generating a plurality of data augmented images by performing a plurality of different data augmentations on the site images;
calculating feature information corresponding to each of the data augmented images by inputting each of the data augmented images into the corresponding feature extraction model; and
concatenating the feature information corresponding to each of the data augmented images and calculating a weight value corresponding to importance of the concatenated feature information for determining the disease.
